(19) United States
(12) Patent Application Publication
(10) Pub. No.: US 2025/0032301 A1
(43) Pub. Date: Jan. 30, 2025

(54) CHASTITY DEVICE AND METHOD OF MONITORING AND CONTROLLING THEREOF

(71) Applicant: HYTTO PTE. LTD., Singapore (SG)

(72) Inventors: Dan LIU, Guangzhou (CN); Jilin QIU, Guangzhou (CN)

(21) Appl. No.: 18/497,894

(22) Filed: Oct. 30, 2023

Related U.S. Application Data
(63) Continuation-in-part of application No. 18/360,230, filed on Jul. 27, 2023.

(51) Int. Cl. A61F 5/37

(52) U.S. Cl. CPC A61F 5/37

(57) ABSTRACT

Embodiments of the present disclosure disclose a chastity device. The chastity device includes a main body, a lock structure, and a restraining member. The main body covers a genital part of a wearer and configured to constrain the genital part at least to receive an external stimulus. The lock structure is mounted to the main body. The restraining member is coupled to the lock structure and is configured to be worn by the wearer at least to constrain movement of the main body. The lock structure is configured to adjust tightness of the restraining member.

### TECHNICAL FIELD

The present disclosure relates to a chastity device to be worn by humans.

### BACKGROUND

A chastity device is used to prevent a wearer from engaging in certain types of sexual activity without the permission of a monitor. The wearer (male or female) may wear the chastity device in several instances. In one instance, the chastity device may be worn by the wearer for not having sex with anyone. In another instance, the chastity device may be worn by the wearer for having sex only with a pre-defined person (e.g., a husband or a wife).

Typically, the chastity device is provided with a locking/unlocking mechanism. The locking/unlocking mechanism may be operated mechanically or electronically. If the chastity device is provided with a mechanically operated locking/unlocking mechanism, a key used for locking/unlocking purposes is often held by the monitor. The monitor may lock/unlock the chastity device through the key, based on mutually agreed discipline between the wearer and the monitor. However, there are chances that the key being used to lock/unlock the chastity device may get misplaced during an emergency. Specifically, due to the misplacing of the key by the monitor, the emergency unlocking of the wearer's chastity device is difficult, and therefore, forceful unlocking of the chastity device is needed in the emergency.

Further, the locking/unlocking mechanism of some of the chastity devices is designed in a manner that the locking/unlocking can be operated electronically through a remote-control application. Such a locking/unlocking mechanism of the chastity device is communicably (e.g., wirelessly) operated by a suitable electronic device of the monitor (e.g., a smartphone). The electronic device eases the locking/unlocking functionally of the chastity device compared to the above-discussed physically operated locking/unlocking mechanism. However, the electronic device configured to operate the locking/unlocking functionality of the chastity device also has some limitations. For example, the electronic device of the monitor can only be operated in a predefined operating range of the chastity device and may get disabled beyond the operating range. Therefore, the electronic device of the monitor has to be operated within the operating range of the chastity device. Also, there is a chance that the electronic device may run out of electrical power on some occasions and thus, may face an operating issue in an emergency. As a result, a forceful unlocking of the chastity device would be needed. However, the forceful unlocking of the chastity device in an emergency involves a risk of injury to the wearer and/or damage to the chastity device.

Therefore, there exists a need to develop a structural mechanism for a chastity device in a manner that does not suffer from the aforementioned deficiencies.

### SUMMARY

Various embodiments of the present disclosure disclose a chastity device that is configured to be monitored and controlled in an emergency.

According to an aspect of the present disclosure, there is provided a chastity device. The chastity device includes a main body, a lock structure, and a restraining member. The main body covers a genital part of a wearer and is configured to constrain the genital part at least to receive an external stimulus. The restraining member is coupled to the lock structure and is configured to be worn by the wearer at least to constrain movement of the main body. The lock structure is configured to adjust tightness of the restraining member.

In an aspect, the lock structure is mounted to the main body.

In an aspect, the restraining member includes a first restrainer and a second restrainer. A first end of first restrainer is fixedly connected to a proximal end portion of the main body and a second end of first restrainer is fixedly connected to the lock structure. A first end of second restrainer is fixedly connected to the proximal end portion of the main body and a second end of second restrainer is removably connected to the lock structure.

In an aspect, the lock structure includes a pressing unit, a locking plate, and a securing member. The locking plate is adapted to constrain a movement of the restraining member in response to application of a pressing force by the pressing unit. The securing member is adapted to engage the locking plate while constraining the movement of the restraining member.

In an aspect, the restraining member is formed with patterns and a bottom side of the locking plate is formed with complementary patterns. The complementary patterns are adapted to be engaged with the patterns of the restraining member and to constrain the movement of the restraining member.

In an aspect, the pressing unit includes a motor having a motor shaft, a cam mounted to the motor shaft, and a locking block including a first end portion and a second end portion. The cam is configured to rotate at a predetermined angle corresponding to a rotational angle of the motor shaft. The first end portion is secured to the cam and the second end portion is oscillatory coupled to a top side of the locking plate. The second end portion is configured to apply pressing force to constrain the movement of the restraining member.

In an aspect, the pressing unit includes an electromagnetic lock. The electromagnetic lock includes a motor and an electromagnetic pressure sheet electromagnetically connected to the motor. The electromagnetic pressure sheet is configured to apply the pressing force on the locking plate to constrain the movement of the restraining member.

In an aspect, the lock structure includes the motor, the motor shaft of the motor, a driver shaft, a gearbox, and a limiting member. The gearbox connects the motor shaft with a proximal end of the drive shaft. The gearbox is configured to control a rotational speed of the driver shaft. The limiting structure is rotatably connected to a distal end of the driver shaft. The limiting member is configured to constrain the movement of the restraining member during a non-use state, and the limiting member pulls the restraining member during a use state

In an aspect, the limiting member is a gear, the gear formed with complementary patterns adapted to be engaged with the patterns of the restraining member.

In an aspect, the motor shaft and the driving shaft are orthogonally coupled with each other.

In an aspect, the restraining member is formed with at least one notch adapted to tear the restraining member.

In an aspect, the restraining member is made up of a flexible material.

In an aspect, the main body includes a urination hole configured to allow the urination of the wearer, a shielding member covering the urination hole at least during a non-use state of the urination hole, and a locking unit coupled to the shielding member and configured to limit an opening and closing of the urination hole.

In an aspect, the locking unit is one of a mechanical lock and an electronic lock.

In an aspect, the chastity device further includes a sensing module, an electronic controller, and a memory unit. The memory unit is operably connected to the electronic controller, the memory unit includes machine-readable instructions, that when executed by the electronic controller, enable the electronic controller to: generate a report signal indicative of a change in a wearing state of the chastity device, in response to an input signal received from the sensing module installed with the chastity device and transmit the report signal to a monitor client device associated with a monitor of the chastity device.

In an aspect, the sensing module is associated with at least one of the main body, the lock structure, and the restraining member. The sensing module is configured at least to detect the wearing state of the chastity device and measure tightness of the restraining member.

In an aspect, the sensing module includes a conductive material associated with the restraining member. The conductive material is disposed within at least a part of the restraining member at least to build a detection circuit therein, indicative of the wearing state of the chastity device.

In an aspect, the restraining member includes a first end and a second end. One of the first end and the second end is configured to pass through the lock structure and coupled to one of the second end and the first end, thereby forming a ring-like structure adapted to be worn by the wearer.

In an aspect, the restraining member is an open-close type restraining member.

In an aspect, the chastity device is one of a male chastity device or a female chastity device.

### BRIEF DESCRIPTION OF THE DRAWINGS

The following detailed description of illustrative embodiments is better understood when read in conjunction with the appended drawings. To illustrate the present disclosure, exemplary constructions of the disclosure are shown in the drawings. However, the present disclosure is not limited to a specific device, or a tool and instrumentalities disclosed herein. Moreover, those in the art will understand that the drawings are not to scale.

FIG. 1A is an example representation of an environment related to at least some examples of the present disclosure;

FIG. 1B illustrates an alternative connection arrangement between a chastity device, a wearer client device, and a monitor client device, in accordance with an embodiment of the present disclosure;

FIG. 2 is a block diagram of a computing device, in accordance with an embodiment of the present disclosure;

FIG. 3A illustrates a first side perspective view of a male chastity device, in accordance with one embodiment of the present disclosure;

FIG. 3B illustrates a second side perspective view of the male chastity device, in accordance with one embodiment of the present disclosure;

FIG. 3C illustrates a side view of the male chastity device, in accordance with one embodiment of the present disclosure;

FIG. 3D illustrates a side view of the male chastity device, in accordance with another embodiment of the present disclosure;

FIG. 4A illustrates a perspective view of a female chastity device, in accordance with an embodiment of the present disclosure;

FIG. 4B illustrates a front view of the female chastity device of FIG. 4A, in accordance with an embodiment of the present disclosure;

FIG. 5A illustrates a perspective view of a lock structure along with other components, in accordance with one embodiment of the present disclosure;

FIG. 5B illustrates a perspective view of the lock structure without housing, in accordance with one embodiment of the present disclosure;

FIG. 5C illustrates an exploded view of the lock structure of FIG. 5A, in accordance with one embodiment of the present disclosure;

FIG. 6A illustrates a perspective view of the lock structure, in accordance with another embodiment of the present disclosure;

FIG. 6B illustrates an exploded view of the lock structure of FIG. 6A, in accordance with another embodiment of the present disclosure; and

FIG. 7 illustrates an exploded view of the lock structure, in accordance with yet another embodiment of the present disclosure.

The drawings referred to in this description are not to be understood as being drawn to scale except if specifically noted, and such drawings are only exemplary in nature.

### DETAILED DESCRIPTION

In the following description, for purposes of explanation, numerous specific details are set forth in order to provide a thorough understanding of the present disclosure. It will be apparent, however, to one skilled in the art that the present disclosure can be practiced without these specific details. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

Reference in this specification to “one embodiment” or “an embodiment” means that a particular feature, structure, or characteristic described in connection with the embodiment is included in at least one embodiment of the present disclosure. The appearances of the phrase “in an embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

Moreover, although the following description contains many specifics for the purposes of illustration, anyone skilled in the art will appreciate that many variations and/or alterations to said details are within the scope of the present disclosure. Similarly, although many of the features of the present disclosure are described in terms of each other, or in conjunction with each other, one skilled in the art will appreciate that many of these features can be provided independently of other features. Accordingly, this description of the present disclosure is set forth without any loss of generality to, and without imposing limitations upon, the present disclosure.

### Overview

Embodiments of the present disclosure provide a chastity device to be worn by a wearer and a method of monitoring and controlling the chastity device. The chastity device includes an electronic controller that detects/changes a wearing state of the chastity device on receiving an input signal from a peripheral device. The peripheral device may be selected from a group consisting of input devices, a communication interface, and a sensing module. On changing the wearing state of the chastity device, the electronic controller generates a report signal indicating the change in the wearing state and transmits the report signal to a monitor client device associated with a monitor. For example, the sensing module may generate the input signal in response to the physiological characteristics of the wearer. The physiological characteristics may include, for example, the size of the genitals, the presence of an injury near the groin area, or some other physical ailment. The sensing module may also determine other parameters such as the tension value of the tension in belt of the chastity device and include the tension value in the input signal.

Alternately, the communication interface may generate the input signal on receipt of a control signal from the monitor client device or a wearer client device associated with the wearer. In that regard, the wearer client device may be able to generate and transmit the control signal to the electronic controller only after receiving an authorization signal from the monitor client device. In another scenario, the input devices may include lock switches, such as a first lock switch and a second lock switch, and the input devices may generate the input signal on the actuation of one or more of the first lock switch and the second lock switch.

In response to the receipt of the input signal, the electronic controller may then operate a hardware module of the chastity device to change the wearing state of the chastity device. In that regard, before operating the hardware module, the electronic controller may perform several verification steps. For example, the electronic controller may compare reported operating parameters included in the input signal with preset input parameters associated with the hardware module. The reported operating parameters may include power parameters such as the State of Charge (SoC) of a battery of the chastity device, a time parameter such as the duration elapsed since the chastity device was last engaged, and a network parameter such as a Service Set Identifier (SSID) of a network to which the chastity device may be connected. The reported operating parameters may also include information associated with a second hardware module of a second chastity device. The associated information may include the distance of the second chastity device from the hardware module, network parameters, power parameters, and time parameters of the second chastity device.

In that regard, the report signal may report the locking and unlocking of the chastity device and may be accompanied by an electronic message. The electronic message may include details of a current wearing state of the chastity device. Further, the electronic message may also include a wearing state change time instance information, the wearing state of the chastity device before and after the instance of the wearing state change, the mode of change of the wearing state, etc. In addition to getting delivered to the monitor client device, the electronic message is also stored in a storage device.

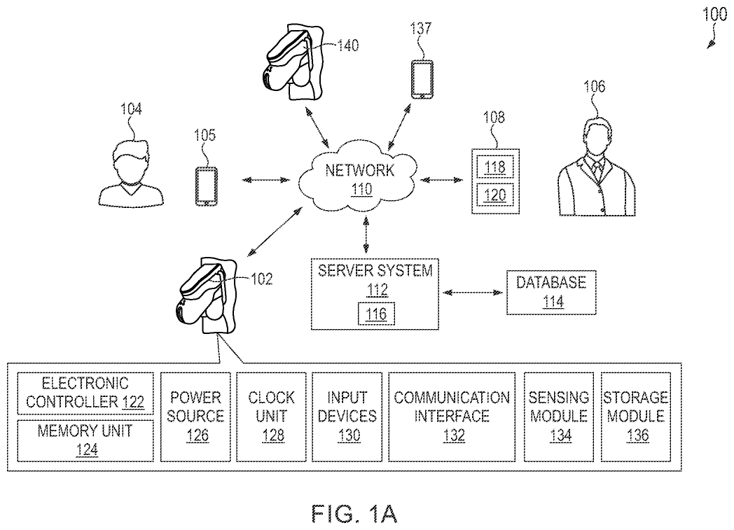

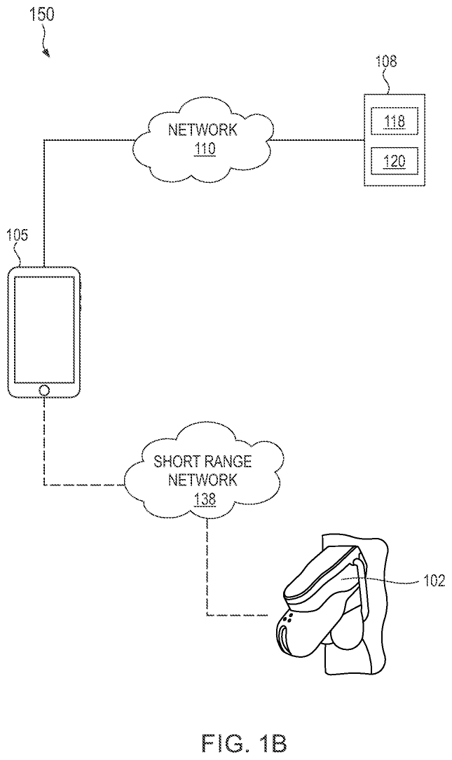

Various example embodiments of the present disclosure are described hereinafter with reference to FIG. 1A to FIG. 7.

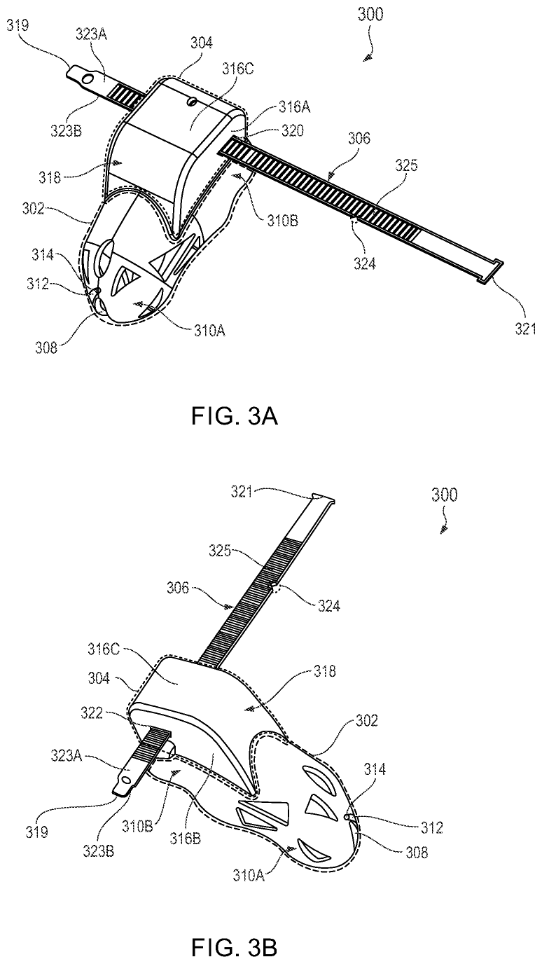

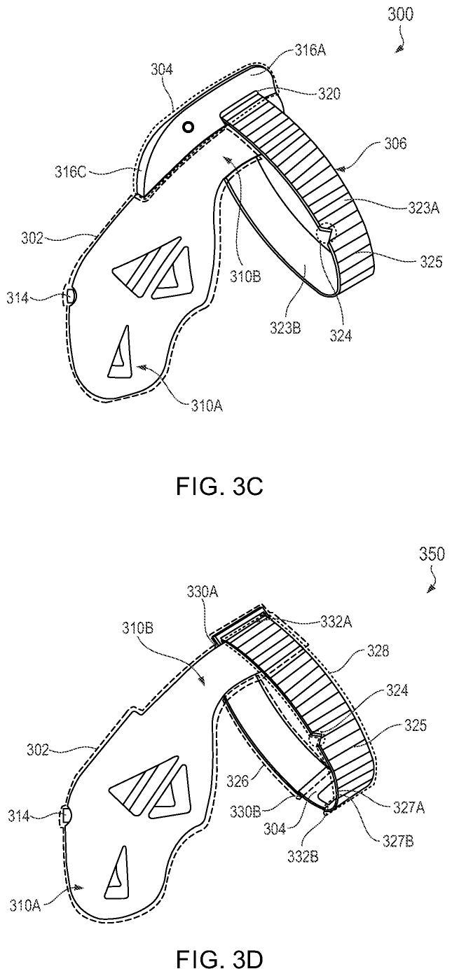

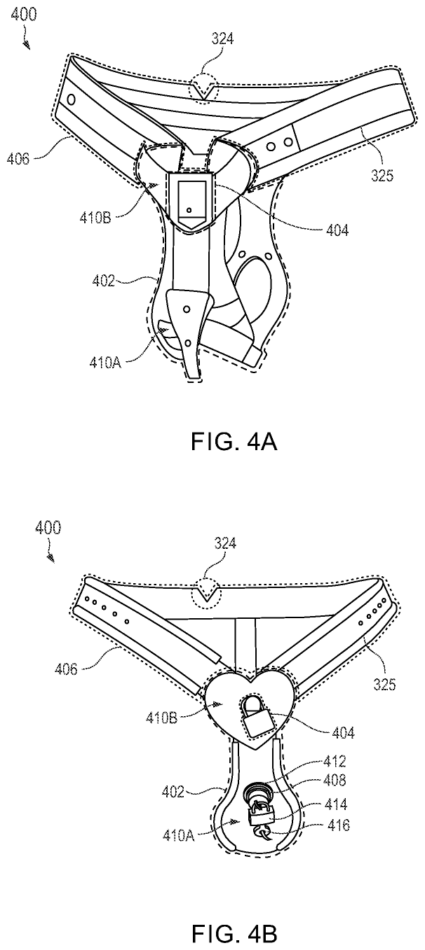

FIG. 1A is an example representation of an environment 100 related to at least some examples of the present disclosure. The environment 100 includes a chastity device 102 designed to be worn by a wearer 104. The chastity device 102 includes a plurality of mechanical and electronic components that prevent the sexual activities of the wearer 104. More specifically, the chastity device 102 worn by the wearer 104 is configured to constrain the wearer 104 from engaging in certain types of sexual activities or having erotic thoughts without the permission of a monitor 106. Depending upon the type of the wearer 104, the chastity device 102 can be designed for both males and females. The chastity device 102 designed for males is referred to as “a male chastity device” (shown in FIGS. 3A to 3D), and the chastity device 102 designed for females is referred to as “a female chastity device” (shown in FIGS. 4A and 4B). In the illustrated embodiment, the wearer 104 of the chastity device 102 is a boy and the monitor 106, monitoring and controlling the chastity device 102, is a person who may be a guardian of the boy or the wearer 104. The guardian (i.e., the monitor 106) restricts the boy or the wearer 104 from engaging in sexual activities (e.g., intercourse, masturbation, etc.) by means of the chastity device 102.

In the illustrated embodiment, the monitor 106 remotely controls and operates the chastity device 102, by providing an electronic input using a monitor client device 108. As shown, the chastity device 102 and the monitor client device 108 are connected with each other via a network 110. The network 110 may include at least one of a Local Area Network (LAN), a Wide Area Network (WAN), a Light Fidelity (Li-Fi) network, a Metropolitan Area Network (MAN), a fiber-optic network, a coaxial cable network, an internet, a satellite network, an Infrared (IR) network, a Radio Frequency (RF) network, a virtual network, or any combination thereof.

FIG. 1B illustrates an alternative connection arrangement 150 between the chastity device 102, the wearer client device 105, and the monitor client device 108, in accordance with an embodiment of the present disclosure. The chastity device 102 may be connected with the monitor client device 108 through the wearer client device 105. In that regard, the chastity device 102 may be connected with the wearer client device 105 through a short-range network 138, such as Bluetooth or Near Field Communication (NFC), and exchange messages with the wearer client device 105 through the short-range network 138. Further, the wearer client device 105 may further connect to the monitor client device 108 through the network 110. In that regard, the wearer client device 105 may act as an intermediary between the chastity device 102 and the monitor client device 108.

The electronic input provided by the monitor 106 through the monitor client device 108 may include but is not limited to, enabling or disabling the operation of the plurality of mechanical and electronic components of the chastity device 102. In a non-limiting example, the monitor 106 may use an interactive standalone application acting as client software and enabled in the monitor client device 108 to provide the electronic input to the chastity device 102 through the network 110. The monitor client device 108 adapted to be operated by the monitor 106 may be any electronic device such as, but not limited to, a smartphone, a laptop, a tablet device, a Personal Computer (PC), a Personal Digital Assistance (PDA), and wearable devices. In the illustrated configuration, the monitor client device 108 is the smartphone that includes the plurality of standalone applications developed to monitor and control the operation of the chastity device 102 worn by the wearer 104 by providing the electronic input to the chastity device 102 via the network 110. In an embodiment, when the chastity device 102 worn by the wearer 104 comes in contact with a pre-defined network area (e.g., home, office, etc.), an electronic controller 122 generates an input signal. Further, the electronic controller 122 transmits the generated input signal to the monitor client device 108, indicating that the chastity device 102 worn by the wearer 104 comes in contact with the pre-defined network area.

In one embodiment, when the chastity device 102 is connected to the monitor client device 108, a message is sent from the chastity device 102 to the monitor client device 108. The same message may then be transmitted from the wearer client device 105 to the monitor client device 108. Alternately, the wearer client device 105 may generate a new message in response to the message received from the chastity device 102 or otherwise and transmit the new message to the monitor client device 108. The message sent to the monitor client device 108 contains information, such as network identity (ID), Internet protocol (IP) address, public encryption key, etc. about the chastity device 102. Alternatively, the chastity device 102 sends the message directly to the monitor client device 108 when the preset conditions are met. The preset conditions may include the monitor client device 108 having a unique registration ID, IP address, or public encryption key and encrypted handshake message that the chastity device 102 may utilize to validate the monitor client device 108. In addition to other constructional features that will be discussed in the following discussion, the chastity device 102 further includes an electronic controller 122, a memory unit 124, a communication interface 132, and a sensing module 134. As an example, the electronic controller 122 may be embodied as one or more of various processing devices, such as a coprocessor, a microprocessor, a controller, a digital signal processor (DSP), a processing circuitry with or without an accompanying DSP, or various other processing devices including integrated circuits such as, for example, an application specific integrated circuit (ASIC), a field programmable gate array (FPGA), a microcontroller unit (MCU). a hardware accelerator, a special-purpose computer chip, integrated circuits, or the like.

The memory unit 124 may be a volatile storage memory, such as Static Random Access Memory (SRAM) and Dynamic Random Access Memory (DRAM) of types such as Asynchronous DRAM, Synchronous DRAM, Double Data Rate SDRAM, Rambus DRAM, and Cache DRAM, etc. The chastity device 102 may also include a storage module 136. The storage module 136 may be a non-volatile storage memory such as EPROM, EEPROM, flash memory, or the like. The memory unit 124 includes machine-readable instructions for execution by the electronic controller 122 for controlling the operation of the chastity device 102. The communication interface 132 may be a device or a module enabling direct connectivity via wires and connectors such as USB, HDMI, VGA, or wireless connectivity such as Bluetooth or Wi-Fi or Local Area Network (LAN) or Wide Area Network (WAN) implemented through TCP/IP, IEEE 802.x, GSM, CDMA, LTE, or other equivalent protocols. The sensing module 134 may include several sensors for sensing a state of the chastity device 102. For example, the sensing module 134 may include one or more sensors for sensing tension in a belt of the chastity device 102, determining if the chastity device 102 is engaged or disengaged. Further, the sensing module 134 may also include a voltage sensor such as a potentiometer to measure voltage in the internal circuitry of the chastity device 102. Other sensors that may form a part of the sensing module 134 may include pressure sensors, proximity sensors, optical sensors, etc.

Further, the illustrated embodiment of the environment 100 includes a server system 112 and a database 114. The server system 112 may be deployed as a standalone server or can be implemented in the cloud as Software as a Service (SaaS). The server system 112 provides or hosts an application 116 for, inter alia, managing unique code generation and enabling and validating user reviews. Furthermore, the monitor client device 108 includes a client processor 118 and a client memory unit 120. The client memory unit 120 is operably connected to the client processor 118. The client memory unit 120 includes machine-readable instructions that when executed by the client processor 118, enable the client processor 118 to, inter alia, provide a control signal to the electronic controller 122 for change in the wearing state of the chastity device 102.

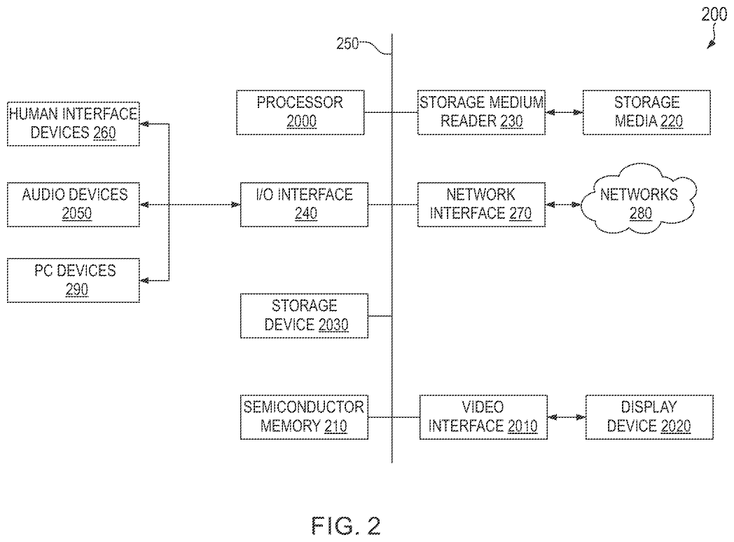

FIG. 2 is a block diagram of a computing device 200, in accordance with an embodiment of the present disclosure. In an embodiment, the computing device 200 takes the form of the server system 112 described in FIG. 1. In this manner, the computing device 200 is adapted to include functionality for communication with the network 110, storage capability (such as the database 114) for storing the data exchanged between the chastity device 102 and the monitor client device 108.

However, it should be noted that the monitor client device 108 of the monitor 106 may also be depicted as the computing device 200. In this manner, the computing device 200 may include differing technical integers, such as a display device 2020 electronically connected to a video interface 2010, one or more human interface devices 260, and the like. In other words, the technical integers of the computing device 200 shown in FIG. 2 are examples only, and variations, adaptations and the like may be made thereto within the purposive scope of the embodiments described herein and having regard for the particular application of the computing device 200.

In particular, the steps of methods for monitoring and controlling the chastity device 102, as described in further detail below, may be implemented as computer program code instructions executable by the computing device 200. The computer program code instructions are executable by the computing device 200. The computer program code may be formed in the form of instruction libraries, such as dynamic link libraries (DLL), wherein each of the libraries performs one or more steps of the methods as would be discussed in the following discussion. Additionally, a subset of one or more libraries may perform graphical user interface tasks relating to the steps of the methods.

The computing device 200 further includes a semiconductor memory 210 including volatile memory such as Random Access Memory (RAM) or Read Only Memory (ROM). The semiconductor memory 210 may include either RAM or ROM or a combination of RAM and ROM. Furthermore, the computing device 200 includes a computer program code storage medium reader 230 for reading the computer program code instructions from computer program code storage media 220. The storage media 220 may be optical media such as CD-ROM disks, magnetic media such as floppy disks and tape cassettes, or flash media such as SUB memory sticks.

Moreover, the computing device 200 includes an I/O interface 240 for communicating with one or more peripheral devices (not shown in FIG. 2). The I/O interface 240 may offer both serial and parallel interface connectivity. For example, the I/O interface 240 may include a Small Computer System Interface (SCSI), and Universal Serial Bus (USB). The I/O interface 240 may also communicate with one or more human interface devices 260 such as keyboards, pointing devices, such as Recommended Standard 232 (RD-2332) interface, for interfacing the computing device 200 with one or more personal computer (PC) devices 290. The I/O interface 240 may also include an audio interface for communicating audio signals to one or more audio devices 2050, such as a speaker or a buzzer.

The computing device 200 also includes a network interface 270 for communicating with one or more computer networks 280, such as the network 110. Computer program code instructions may be loaded into the storage device 2030 from the storage media 220 using the storage medium reader 230 or from the network 110 using the network interface 270. During the bootstrap phase, an operating system and one or more software applications are loaded from the storage device 2030 into the semiconductor memory 210. During the fetch-decode-execute cycle, a processor 2000 fetches computer program code instructions from the semiconductor memory 210, decodes the instructions into machine code, executes the instructions, and stores one or more intermediate results in the semiconductor memory 210.

In this manner, the instructions stored in the semiconductor memory 210, when retrieved and executed by the processor 2000, may configure the computing device 200 as a special-purpose machine that may perform the functions described above. The computing device 200 also includes a communication bus subsystem 250 for interconnecting the various devices described above. The communication bus subsystem 250 may offer parallel connectivity such as Industry Standard Architecture (ISA) conventional Peripheral Component Interconnect (PCI) and the like or serial connectivity such as PCI Express (PCIe), Serial Advanced Technology Attachment (Serial ATA), and the like.

FIG. 3A illustrates a perspective view of a first side of a male chastity device 300, in accordance with one embodiment of the present disclosure. FIG. 3B illustrates a second side perspective view of the male chastity device 300, in accordance with one embodiment of the present disclosure. FIG. 3C illustrates a side view of the male chastity device 300, in accordance with one embodiment of the present disclosure. The male chastity device 300 disclosed herein is designed to prevent the wearer 104 (i.e., the male wearer) from engaging in certain types of sexual behavior without the permission of the monitor 106. The chastity device 102 includes a main body 302, a lock structure 304, and a restraining member 306.

The main body 302 is configured to constrain the wearer 104 (i.e., the male wearer) from engaging in certain types of sexual activities or having erotic thoughts without the permission of the monitor 106. More specifically, the main body 302 is designed to cover a genital part (i.e., penis) of the wearer 104, and to prevent the wearer 104 from doing sexual activities. The design of the main body 302 is such that a flaccid state of the genital part (e.g., penis) of the wearer 104 can be secured in a safe manner. It is to be noted that the size of the main body 302 may not be limited to one particular value and may be of any size, depending upon the size of the penis of the wearer 104. The main body 302 may be made up of a suitable rigid lightweight material that does not cause injury or infection to the penis of the wearer 104. In a non-limiting example, rigid lightweight materials such as silicon, borosilicate glass, Lucite, stainless steel, etc., can be used to fabricate the main body 302. The geometrical configuration of the main body 302 is such that the attachment and detachment capability of the main body 302 provides case of cleaning the male chastity device 300. The case of cleaning the main body 302 would assist in maintaining a specific hygiene level in the male chastity device 300.

Further, the main body 302 is formed with a urination hole 308 (shown in FIGS. 3A and 3B). As shown, the urination hole 308 is formed in a distal end portion 310A of the main body 302. The urination hole 308 is configured to allow urination of the wearer 104. The main body 302 also includes a shielded member 312 (shown in FIGS. 3A and 3B) configured to cover the urination hole 308 at least during a non-use state of the urination hole 308. The shielded member 312 may form a shape complementary to the shape of the urination hole 308 and functions using a locking unit 314. The locking unit 314 is configured to limit the opening and closing of the shielded member 312. In one embodiment, the locking unit 314 may be configured with a mechanical lock that operates manually by the wearer 104 or the monitor 106. The locking unit 314 can be operated by the wearer 104 or the monitor 106 through a mechanical key (not shown in FIGS. 3A-3C). The locking unit 314 may be provided with a keyhole adapted to receive the mechanical key and to unlock the locking unit 314. The unlocking of the locking unit 314 allows the opening of the shielded member 312 to allow the urination of the wearer 104. In another embodiment, the locking unit 314 may be configured with an electronic lock that operates electronically by the wearer 104 using the wearer client device 105 or by the monitor 106 using the monitor client device 108. The unlocking of the locking unit 314 allows the opening of the shielded member 312 to allow the urination of the wearer 104.

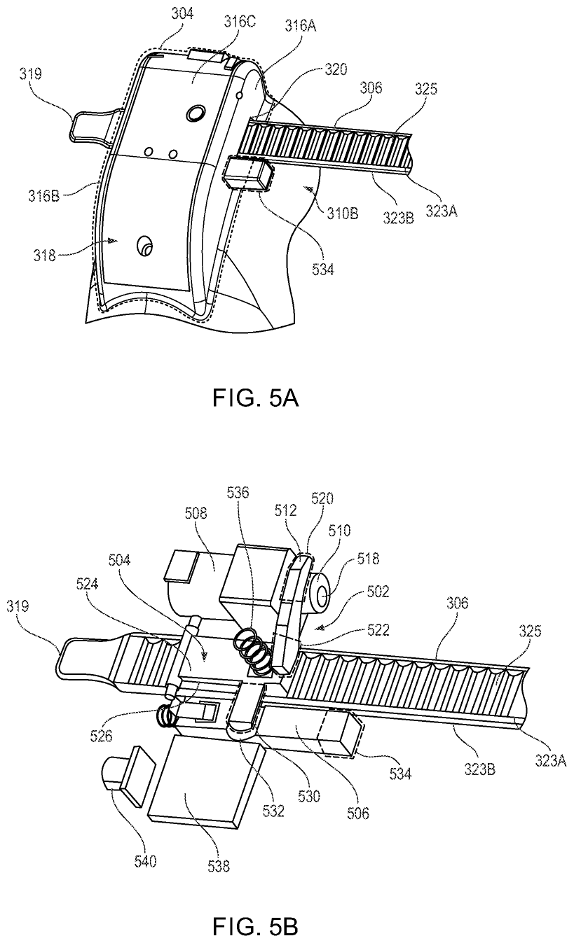

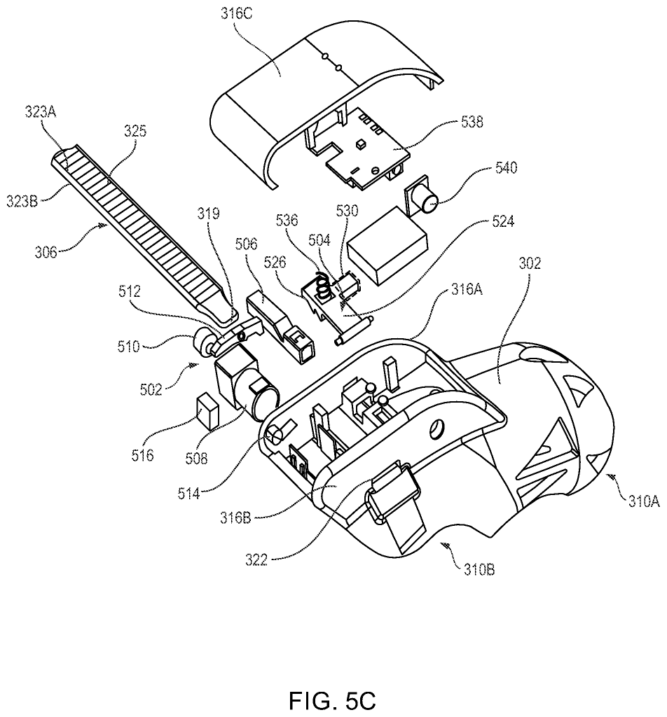

In the illustrated embodiment, the lock structure 304 (shown as covered in dashed area) is fixedly mounted to a proximal end portion 310B of the main body 302. The lock structure 304 includes a first side cover 316A (shown in FIGS. 3A and 3C), a second side cover 316B (shown in FIG. 3B), and a top cover 316C (shown in FIGS. 3A and 3B) connecting the first side cover 316A and the second side cover 316B. The first side cover 316A, the second side cover 316B, and the top cover 316C collectively form a housing 318. The housing 318 is configured to cover the plurality of mechanical and electronic components disposed therein. The plurality of mechanical and electronic components of the lock structure 304 are illustrated and discussed in further detail with respect to FIGS. 5A-5C to 7 in the following discussion. In an embodiment, the housing 318 is constructed in a manner to be split into at least three parts. The split parts (i.e., the first side cover 316A, the second side cover 316B, and the top cover 316C) may be assembled through suitable fastening elements. In a non-limiting example, the glue may be applied on the border of contacting surfaces of the first side cover 316A, the second side cover 316B, and the top cover 316C to form a unitary structure of the housing 318. It should be noted that the shape of the housing 318 can vary in other embodiments, based on the design configuration of the chastity device 102. Also, it is to be noted that the lock structure 304 may or may not be mounted to the main body 302. In another embodiment of the present disclosure, the lock structure 304 can be mounted to the restraining member 306 (shown in FIG. 3D).

The first side cover 316A is formed with a first opening 320 (shown in FIG. 3A). The first opening 320 is adapted at least to enable the restraining member 306 to pass therethrough. Likewise, the second side cover 316B is formed with a second opening 322 (shown in FIG. 3B). The second opening 322 is adapted to allow the restraining member 306 to pass therethrough. A shape of each of the first opening 320 and the second opening 322 is in line with the cross-sectional shape of the restraining member 306. In the illustrated configuration, each of the first opening 320 and the second opening 322 is formed with a rectangular shape that is in line with a rectangular cross-section of the restraining member 306. However, in another configuration, the restraining member 306 can be formed with a circular cross-section, and correspondingly, the first opening 320 and the second opening 322 would be formed with a circular shape.

The restraining member 306 is designed to pass through the first opening 320 and the second opening 322 and is configured to be worn by the wearer 104 at least to constrain a movement of the main body 302. A tightness of the restraining member 306 is adjusted through the lock structure 304. The wearer 104 or the monitor 106 can pull the restraining member 306 in one direction to form a ring-like structure. The design of the male chastity device 300 is such that the main body 302 covers the penis and the ring-like structure formed by the restraining member 306 surrounds a root of the penis and a scrotum. The restraining member 306 includes a first end 319 and a second end 321. One of the first end 319 and the second end 321 is configured to pass through the first opening 320 and the second opening 322 of the lock structure 304 and coupled to one of the second end 321 and the first end 319, thereby forming the ring-like structure. In one configuration, the first end 319 can be passed through the first opening 320 and the second opening 322 of the lock structure 304 and coupled to the second end 321 to form the ring-like structure. In another configuration, the second end 321 can be passed through the first opening 320 and the second opening 322 of the lock structure 304 and coupled to the first end 319 to form the ring-like structure. The restraining member 306 forming the ring-like structure is an open-close type restraining member that fixes the relative position between the main body 302 and the penis of the male wearer. As a result, the chances of sliding or detachment of the male chastity device 102 without the permission of the monitor 106 is eliminated. The diameter of the ring-like structure can be varied through the lock structure 304. In a non-limiting example, when the first end 319 of the restraining member 306 is pulled in one direction, the diameter of the ring-like structure decreases and the male chastity device 300 fits tighter to the penis of the wearer 104. On the other hand, when the first end 319 of the restraining member 306 is pulled in another direction, the diameter of the ring-like structure increases, and the male chastity device 300 fits looser to the penis of the wearer 104.

A first surface 323A of the restraining member 306 is formed with patterns 325, and a second surface 323B (shown in FIG. 3C) can be formed as a smooth surface. The patterns 325 are adapted to be constrained by one or more components of the lock structure 304. In the illustrated embodiment, the patterns 325 having a shape of teeth profiles are formed along the width of the restraining member 306. However, in other embodiments, the patterns 325 such as, but not limited to, knuckle patterns, textured patterns, etc., can be formed on the first surface 323A of the restraining member 306.

Further, the restraining member 306 is formed with at least one notch 324 (also referred to as “notch 324”). The notch 324 is formed perpendicular to the length of the restraining member 306 (i.e., along the width of the restraining member 306). The notch 324 reduces at least a localized bending resistance which cases the tearing of the restraining member 306 into two pieces during an emergency situation. In the illustrated example, the notch 324 has a V-shape that can tear manually or through a suitable device like a scissor. In other embodiments, the notch 324 can have other shapes like a groove, a semi-circular, a trapezoidal, etc., that ease the tearing of the restraining member 306 into two pieces during the emergency situation.

The restraining member 306 used in this embodiment is a belt. The belt is made up of a suitable flexible and soft material (e.g., rubber, leather, etc.) that eliminates the chances of injury causing to the genital part (e.g., penis) of the wearer 104.

Further, the male chastity device 300 includes the sensing module 134 adapted to detect the wearing state of the male chastity device 300. The sensing module 134 is associated with at least one of the main body 302, the lock structure 304, and the restraining member 306, and configured at least to detect the wearing state of the male chastity device 300 and measure the tightness of the restraining member 306. In an embodiment, when the unlocking operation on the restraining member 306 of the male chastity device 300 is detected, the corresponding unlocking information for the operation is generated. The unlocking operation causes the restraining member 306 of the male chastity device 300 to switch from the locked state to the unlocked state. The sensing module 134 (e.g., tension load cell) first measures the tightness level of the restraining member 306 of the male chastity device 300, then the sensing module 134 generates an image and/or information that represents the tightness level of the restraining member 306 of the male chastity device 300 and sends the image and/or information to the monitor client device 108 over the network 110. The image and/or information may also be stored in the database 114 through the server system 112 for later reference and/or system improvements. The sensing module 134 includes one or more sensors adapted to detect the wearing state of the male chastity device 300.

In one example, different areas of the restraining member 306 are formed of different colors, and the position of the restraining member 306 can be known by identifying the color of the restraining member 306 formed in a particular area through a color sensor (e.g., SPECTRO color sensor).

In another example, different areas of one side of the restraining member 306 are formed with different surface roughness, and the position of the restraining member 306 can be known by identifying the surface roughness of the restraining member 306 formed in a particular area through a suitable sensor like an interferometric sensor.

In another example, a conductive material (e.g., conductive wire) is disposed within the restraining member 306. In one configuration, the conductive material can be disposed within an entire length of the restraining member 306 and passes through both ends of the restraining member 306. When both ends of the conducting wire are connected to each other, a detection circuit is built therein. This indicates that the restraining member 306 is in a connected state, otherwise, in a disconnected state. In another configuration, the conductive material can be disposed within a part of the length of the restraining member 306. When both the ends of the conducting wire disposed within the part of the length of the restraining member 306 are connected to each other, the detection circuit is built therein. This indicates that the restraining member 306 is in a connected state, otherwise, in a disconnected state.

In yet another example, a distance sensor can be disposed on a wear face side of one of the main body 302 or the restraining member 306 to detect the distance of the user. Based on this, it can be judged whether the wearer 104 wore the male chastity device or not.

FIG. 3D illustrates a side view of a male chastity device 350, in accordance with another embodiment of the present disclosure. In this embodiment of the male chastity device 350, the restraining member 306 consists of two independent restraints, a first restraint 326 and a second restraint 328. The first restraint 326 and the second restraint 328 are connected to each other through the lock structure 304. Herein the lock structure 304 is not mounted to the main body 302, instead, the lock structure 304 acts as a connection link between the first restraint 326 and the second restraint 328. In such a configuration of the male chastity device 350, a first end 330A of first restrainer 326 and a first end 332A of second restrainer 328 are fixed to the proximal end portion 310B of the main body 302. In the illustrated example, a first slot is formed on one side of the primal end portion 310B to fixedly secure the first end 330A of the first restrainer 326. Likewise, a second slot is formed on another side of the primal end portion 310B to fixedly secure the first end 332A of the second restrainer 328. Further, a second end 330B of first restraint 326 is fixedly secured to the lock structure 304, whereas, a second end 332B of second restraint 328 is removably secured to the lock structure 304. In other words, the first restraint 326 is fixed and the second restraint 328 is adjustable with respect to the lock structure 304. A mounting arrangement of the first end 330A and the second end 330B of the first restrainer 326 and the first end 332A and the second end 332B of the second restrainer 328 collectively form the ring-like structure. It is to be noted that in another configuration of this embodiment, the second end 332B of the second restraint 328 can be fixedly secured to the lock structure 304, and the second end 330B of first restraint 326 can be removably secured to the lock structure 304. In other words, the second restraint 328 is fixed and the first restraint 326 is adjustable with respect to the lock structure 304.

In yet another configuration, both the second end 330B of the first restrainer 326 and the second end 332B of the second restrainer 328 can be removably secured to the lock structure 304. The lock structure 304 can be detached from the second end 330B of the first restrainer 326 and the second end 332B of the second restrainer 328 during a non-use state of the male chastity device 350. However, during a use state of the male chastity device 350, the lock structure 304 connects the the second end 330B of the first restrainer 326 and the second end 332B of the second restrainer 328 to form the ring-like structure. In such a configuration, the lock structure 304 acts as a removal connecting link between the second end 330B of the first restrainer 326 and the second end 332B of the second restrainer 328. The lock structure 304 may be provided with engaging members adapted to be engaged with complementary engaging members of the second end 330B of the first restrainer 326 and the second end 332B of the second restrainer 328 to form the ring-like structure. In a non-limiting example, the connection between the lock structure 304, the first restrainer 326, and the second restrainer 328 has a zipper-like structure. In such a structure, the lock structure 304 is constructed like a slider of the zipper, and the second end 330B of the first restrainer 326 and the second end 332B of the second restrainer 328 are provided with a plurality of tooth profiles. The sliding motion of the slider (i.e., the lock structure 304) latches every hook of the plurality of tooth profiles provided on the second end 330B of the first restrainer 326 and the second end 332B of the second restrainer 328. The slider can manually lock with the plurality of tooth profiles or can be provided with an automated locking mechanism.

In the illustrated configuration, the tightness of the second restrainer 328 is adjusted through the lock structure 304. The wearer 104 or the monitor 106 can pull the second restrainer 328 in one direction to form a ring-like structure. The diameter of the ring-like structure can be varied through the lock structure 304. In a non-limiting example, when the second restrainer 328 is pulled in one direction, the ring diameter decreases and the male chastity device 350 fits tighter to the penis of the wearer 104. On the other hand, when the second restrainer 328 is pulled in another direction, the ring diameter increases, and the male chastity device 350 fits looser to the penis of the wearer 104.

Herein, a first surface 327A of the second restrainer 328 is formed with the patterns 325, and a second surface 327B of the second restrainer 328 is formed with a smooth surface. Further, both the surfaces of the first restrainer 326 are formed with smooth surfaces. The patterns 325 formed on the first surface 327A are adapted to be constrained by the lock structure 304. In the illustrated embodiment, the patterns 325 on the first surface 327A of second restrainer 328 are protruded members positioned at the predetermined gap along a width of the second restrainer 328. However, in other embodiments, other types of patterns 325 such as, but not limited to, knuckle patterns, textured patterns, etc., can be formed on the first surface 327A of the second restrainer 328.

In the illustrated embodiment, the first restraint 326 and the second restraint 328 are belts. The belts are made up of a suitable flexible and soft material (e.g., rubber, leather, etc.) that eliminates the chances of injury causing to the genital part (e.g., penis) of the wearer 104.

FIG. 4A illustrates a perspective view of a female chastity device 400, in accordance with an embodiment of the present disclosure. FIG. 4B illustrates a front view of the female chastity device 400 of FIG. 4A, in accordance with an embodiment of the present disclosure. In comparison with the male chastity device 300 shown in FIGS. 3A to 3D, a geometrical configuration of a main body 402 and a restraining member 406 of the female chastity device 400 differs from the corresponding components of the male chastity device 300, due to a body structure of the female. However, the operating features of each of the main body 402 and the restarting member 406 remain the same.

The main body 402 is configured to constrain the wearer 104 (i.e., the female wearer) from engaging in certain types of sexual activities or having erotic thoughts without the permission of the monitor 106. More specifically, the main body 402 is designed to cover the genital part (i.e., vagina) of the female wearer, and to prevent the wearer 104 from doing sexual activities. The design of the main body 402 is such that the vagina of the female wearer can be covered in a safe manner. Further, it should be noted that the size of the main body 402 may not be limited to one particular value and may be of any size, depending upon the size of the vagina of the female wearer. The main body 402 may be made up of a suitable rigid lightweight material that does not cause injury or infection to the vagina of the female wearer. For example, rigid lightweight materials such as silicon, borosilicate glass, Lucite, stainless steel, etc., can be used to fabricate the main body 402. The geometrical configuration of the main body 402 is such that the attachment and detachment capability of the main body 402 provides case of cleaning the female chastity device 400. The case of cleaning the main body 402 would assist in maintaining a specific hygiene level in the female chastity device 400.

Further, the main body 402 is formed with a female urination hole 408. As shown, the female urination hole 408 (shown in FIG. 4B) is formed on a lower portion 410A of the main body 402. The female urination hole 408 is configured to allow urination of the female wearer. The main body 402 also includes a shielded member 412 configured to cover the female urination hole 408 at least during a non-use state of the female urination hole 408. The shielded member 412 may form a shape complementary to the shape of the female urination hole 408 and function using a locking unit 414. The locking unit 414 is configured to limit the opening and closing of the shielded member 412. In the illustrated embodiment, the locking unit 414 is configured with the mechanical lock that operates manually by the wearer 104 or the monitor 106. The locking unit 414 can be operated by the wearer 104 or the monitor 106 through the mechanical key 416. The locking unit 414 disclosed herein is provided with the keyhole adapted to receive the mechanical key 416 and to unlock the locking unit 414. The unlocking of the locking unit 414 allows the opening of the shielded member 412 to allow the urination of the wearer 104. In another embodiment, the locking unit 414 may be configured with an electronic lock that operates electronically by the wearer 104 using the wearer client device 105 or by the monitor 106 using the monitor client device 108. The unlocking of the locking unit 414 allows the opening of the shielded member 412 to allow the urination of the wearer 104.

In the illustrated embodiment, a lock structure 404 of the female chastity device 400 is fixedly mounted to a top portion 410B of the main body 402. The lock structure 404 may have the same or different geometrical configuration and operating features than that of the lock structure 304 of the male chastity device 300. In one embodiment disclosed in FIG. 4A, the lock structure 404 has the same configuration as that of the lock structure 304 used in the male chastity device 300, and therefore, the geometrical characteristics and operating features of the lock structure 404 are not reiterated here for the sake of brevity. However, in another embodiment disclosed in FIG. 4B, the lock structure 404 has a mechanical configuration, however, the operating features would remain the same.

The restraining member 406 shown in the illustrated configuration is designed to tie to the waist of the female wearer. The restraining member 406 is provided with the patterns. Herein, the patterns 325 consists of longitudinal grooves and plurality of holes in between the longitudinal grooves. Further, the restraining member 406 is configured with the notch 324. The notch 324 is formed perpendicular to a length of the restraining member 406 (i.e., along the width of the restraining member 406). The notch 324 reduces at least a localized bending resistance which cases the tearing of the restraining member 406 into two pieces during an emergency situation. In the illustrated example, the notch 424 has a V-shape that can tear manually or through a suitable device like the scissor. In other embodiments, the notch 324 can have other shapes like a groove, a semi-circular, a trapezoidal, etc., that case the tearing of the restraining member 406 into two pieces during the emergency situation.

However, in another configuration of the female chastity device 400, the restraining member 406 can be designed to tie to the thighs of the female wearer. The restraining member 406 configured to tie to the thighs would have the same configuration as that of the restraining member 406 designed to tie to the waist of the female wearer, and therefore, not reiterated here for the sake of brevity.

FIG. 5A illustrates a perspective view of the lock structure 304 along with other components, in accordance with one embodiment of the present disclosure. FIG. 5B illustrates a perspective view of the lock structure 304 without the housing 318, in accordance with one embodiment of the present disclosure. FIG. 5C illustrates an exploded view of the lock structure 304 of FIG. 5A, in accordance with one embodiment of the present disclosure. The lock structure 304 mainly includes a pressing unit 502, a locking plate 504, and a securing member 506. Referring to FIGS. 5B and 5C, the pressing unit 502 is positioned on one side of the locking plate 504, whereas, the securing member 506 is positioned on another side of the locking plate 504. The pressing unit 502 is designed to apply a pressing force (i.e., a compressive force) on the locking plate 504. The locking plate 504 is configured to constrain the movement of the restraining member 306 using the securing member 506.

The pressing unit 502 includes a motor 508, a cam 510, a locking block 512, and a shrapnel 514. The motor 508 receives power from a power source 516 (shown in FIG. 5C). The motor 508 may be connected to the power source 516 through the electronic controller 122 (not shown in FIGS. 5A to 5C). The power source 516 is configured to provide electrical power to the motor 508 utilized to rotate a motor shaft 518. The power source 202 is capable of providing an alternating current (AC) or a direct current (DC) as per the configuration of the motor 508. In the illustrated configuration, the power source 202 is a battery (e.g., a Lithium-ion battery, Lithium-polymer battery, Nickel-Metal-Hydride battery, etc.) configured to drive the motor 508. The controller controls the power supply to the motor 508. Based on regulating the power supply to the motor 508, the rotational speed of the motor 508 can be changed. In an example, the controller regulates a voltage or a current supplied to the motor 508, which in turn regulates the rotational speed of the motor 508.

The motor 508, disposed within the housing 318, is compact in design and serves the purpose of converting electrical energy into mechanical motion (i.e. movement of the motor shaft 518).

The cam 510 is mounted to the motor shaft 518 of the motor 508 and is configured to rotate at a predetermined angle corresponding to a rotational angle of the motor shaft 518. The cam 510 is a solid structural member constructed as a non-circular profile having a bore designed to accommodate the motor shaft 518. In one configuration, the motor shaft 518 may be fixed within the bore of the cam 510 using a suitable key joint. In another configuration, the motor shaft 518 may fit within the bore of the cam using an interference fit. In other words, the cam 510 may press fit with the motor shaft 518. Based on this arrangement, the relative motion between the motor shaft 518 and the cam 510 can be eliminated. The cam 510 may be constructed to form a circular or non-circular shape. The geometrical profile of the cam 510 is designed to secure a first end portion 520 of the locking block 512.

The first end portion 520 of the locking block 512 is secured to the cam 510 and a second end portion 522 of the locking block 512 is oscillatory connected to the locking plate 504. In other words, the locking block 512 is a connecting link between the cam 510 and the locking plate 504. The first end portion 520 of the locking block 512 can be temporarily or permanently secured to the cam 510. The locking block 512 converts the rotary motion into oscillatory motion. The oscillatory motion of the locking block 512 moves the second end portion 522 upwardly or downwardly by a predetermined height, depending upon the direction of rotation of the motor shaft 518.

When the second end portion 522 moves downwardly by the predetermined height, a surface contact between the second end portion 522 and a part of a top side 524 of the locking plate 504 takes place. The locking plate 504 is mounted to a portion of the restraining member 306. The top side 524 is adapted to receive a pressing force (i.e., compressing force) to constrain an upward movement of the locking part 504. A bottom side 526 of the locking plate is formed with complementary patterns (not shown in FIGS. 5A to 5C) of the patterns 325 of the restraining member 306. The complementary patterns are adapted to be engaged with the patterns 325 of the restraining member 306 and to constrain the movement of the restraining member 306. The shrapnel 514 exerts a downward force on the locking block 512. With this downward force, the blocking block 512 presses down to tighten the locking plate 504.

When the second end portion 522 moves upwardly by the predetermined height, there is no surface contact between the second end portion 522 and the top side 524 of the locking plate 504. In this case, the locking plate 504 moves upwardly by a predefined height, and a considerable clearance between the restraining member 306 and the bottom side 526 is provided. As a result, the restraining member 306 moves through the lock structure 304.

The locking plate 504 includes a protruded member 530. The protruded member 530 is extended outwardly to the other side where the securing member 506 is positioned. The protruded member 530 is shown in FIG. 5B has a half-cylinder shape. The protruded member 530 is extended in a manner that a flat surface is exposed upwardly and a curved surface is exposed downwardly. Further, the length of the protruded member 530 is at least equal to the width of the securing member 506.

The securing member 506 used in the illustrated embodiment is a flat plate formed with a depression 532 along the width direction. The depression 532 forms a shape complementary to the shape of the protruded member 530. In other words, the depression 532 forms the shape of a half-cylindrical channel to slidably secure the protruded member 530. It should be noted that, in another embodiment of the disclosure, the geometrical profiles of the protruded member 530 and the depression 532 can be different such as but not limited to a semi-elliptical, semi-hexagonal, etc. When the protruded member 530 is positioned on the depression 532, the movement of the restraining member 306 is constrained by the locking plate 504. In this case, the pressing unit 502 applies pressing force on the locking plate 504 in a manner that the locking plate 504 cannot be pushed by the securing member 506. As a result, the protruded member 530 cannot be moved out of the depression 532. On the other hand, when the protruded member 530 is slidably moved to a non-depression surface (i.e., the flat surface), the locking plate 504 detaches from the restraining member 306, and thus, the movement of the restraining member 306 is possible.

An extended portion 534 of the securing member 506 is passed through the first side cover 316A along the length direction of the restraining member 306. When the wearer 104 or the monitor 106 pushes the extended portion 534 of the securing member 506, the protruded member 530 slidably detaches from the depression 532 and moves to the flat surface. As a consequence, the locking plate 504 detaches from the restraining member 306, and the restraining member 306 can move in a length direction.

When the protruded member 530 is positioned to the flat surface, and the wearer 104 or the monitor 106 no longer pushes the extended portion 534 of the securing member 506 inwardly, the securing member 506 resets automatically. A compression spring 536 transversely secured to the top side 524 of the locking plate 504. The compression spring 536 may be extended in between the top side 524 of the locking plate 504 and the top cover 316C of the housing 318. The compression spring 536 is secured to the top side 524 in a manner that the axial movement of the locking plate 504 is constrained, which in turn secures the protruded member 530 in the depression 532. In a non-limiting example, upon receiving the input signal from the monitor device 108, the motor 508 rotates the cam 510 clockwise by a predetermined angle. The clockwise rotation of the cam 510 by a predetermined angle drives the locking block 512 from upward wrapping to downward pressing, and the shrapnel 514 exerts a downward force on the locking block 512. With this downward force, the blocking block 512 presses down to tighten the locking plate 504, thereby constraining the inward movement of the extended portion 534 of the locking block 512 required to lift the locking plate 504. An electrical connection of the one or more components discussed above can be operated by a printed circuit board (PCB) 538. The PCB can be switched ON or switched OFF by a PCB switchboard 540.

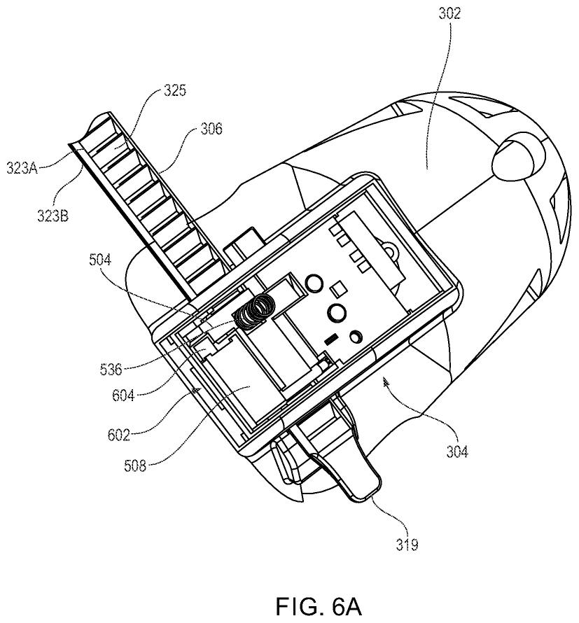

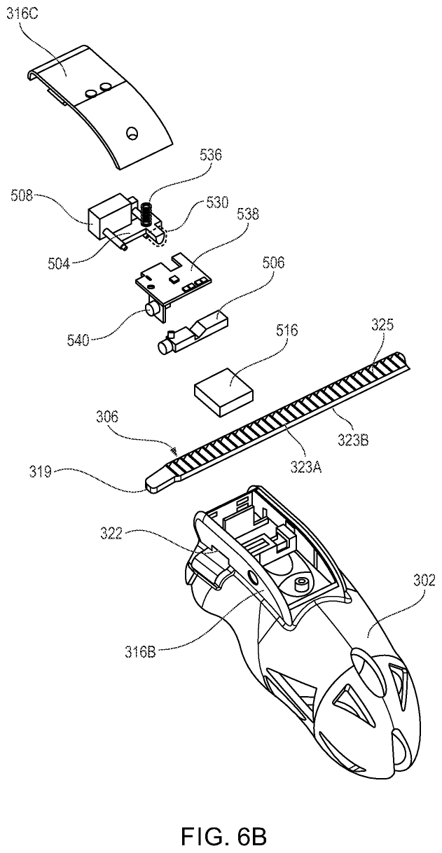

FIG. 6A illustrates a perspective view of the lock structure 304, in accordance with another embodiment of the present disclosure. FIG. 6B illustrates an exploded view of the lock structure 304 of FIG. 6A, in accordance with another embodiment of the present disclosure. In this embodiment, the pressing unit 502 is an electromagnetic lock 602 that includes the motor 508 and an electromagnetic pressure sheet 604. When the movement of the restraining member 306 is to be constrained by the lock structure 304, the electromagnetic sheet applies pressing force on the top side 524 of the locking plate 504. The pressing force constrains the movement of the restraining member 306.

When the male chastity device 300 is in the unlocked state, the motor 508 is energized so that the electromagnetic pressure sheet 604 retracts toward the motor 508, and the electromagnetic pressure sheet 604 moves away from the locking plate 504, so that the locking plate 504 can be lifted. The compression spring 536 connects between the electromagnetic pressure sheet 604 and the motor 508. When the motor 508 is powered off, the force on the electromagnetic pressure sheet 604 will be withdrawn. Due to the spring, the electromagnetic pressure sheet 604 will stretch forward and compress the depression 532 of the locking plate 504 again.

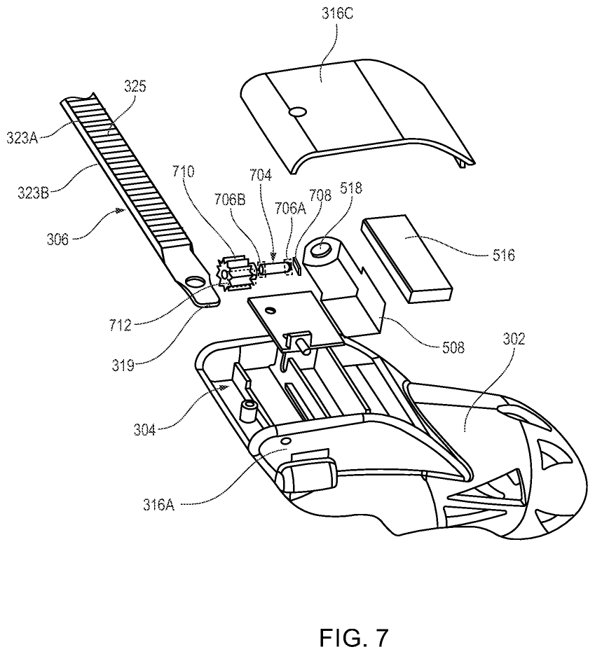

FIG. 7 illustrates an exploded view of the lock structure 304, in accordance with yet another embodiment of the present disclosure. The lock structure 304 includes the motor 508, the motor shaft 518, and a driver shaft 704. A proximal end 706A of the driver shaft 704 is rotatably coupled to the motor shaft 518 through a gearbox 708, and a distal end 706B of the driver shaft 704 is coupled to a limiting member 710. The gearbox 708 is configured to convert an axial rotary motion of the motor shaft 518 into a lateral rotary motion of the driver shaft 704. In other words, the motor shaft 518 and the driver shaft 704 are angularly (i.e., orthogonally or obliquely) coupled with each other using the gearbox 708. In a non-limiting example, the gearbox 708 includes at least a pair of bevel gears (i.e., a gear and a pinion) designed to connect the motor shaft 518 orthogonally with the driver shaft 704.

In the illustrated configuration, the limiting member 710 is a gear formed with the complementary pattern 712 that is adapted to be engaged with the patterns 325 of the restraining member 306, and to constrain the movement of the restraining member 306. When the motor 508 receives the input signal from the controller, a direction of rotation of the motor 508 is controlled and the gearbox 708 controls the rotational speed of the driver shaft 704 onto which the limiting member 710 is fixedly mounted. The limiting member 710 is configured to constrain the movement of the restraining member during a non-use state. When the limiting member 710 is rotated, the pulling of the restraining member 306 takes place. It is to be noted that the operations involved are automated and human intervention is not needed.

Various embodiments of the disclosure, as discussed above, may be practiced with steps and/or operations in a different order, and/or with hardware elements in configurations, which are different from those which are disclosed. Therefore, although the disclosure has been described based on these exemplary embodiments, it is noted that certain modifications, variations, and alternative constructions may be apparent and well within the scope of the disclosure.

Although various exemplary embodiments of the disclosure are described herein in a language specific to structural features and/or methodological acts, the subject matter defined in the appended claims is not necessarily limited to the specific features or acts described above. Rather, the specific features and acts described above are disclosed as exemplary forms of implementing the claims.

1. A chastity device, comprising:
a main body covering a genital part of a wearer and configured to constrain the genital part at least to receive an external stimulus;
a lock structure; and
a restraining member coupled to the lock structure configured to be worn by the wearer at least to constrain a movement of the main body, wherein the lock structure is configured to adjust a tightness of the restraining member.

2. The chastity device as claimed in claim 1, wherein the lock structure is mounted to the main body.

3. The chastity device as claimed in claim 1, wherein the restraining member comprises:
a first restrainer, a first end of first restrainer fixedly connected to a proximal end portion of the main body and a second end of first restrainer fixedly connected to the lock structure; and
a second restrainer, a first end of second restrainer fixedly connected to the proximal end portion of the main body and a second end of second restrainer removably connected to the lock structure.

4. The chastity device as claim in claim 1, wherein the lock structure comprises:
a pressing unit;
a locking plate adapted to constrain a movement of the restraining member in response to application of a pressing force by the pressing unit; and
a securing member adapted to engage the locking plate while constraining the movement of the restraining member.

5. The chastity device as claimed in claim 4, wherein the restraining member is formed with patterns and a bottom side of the locking plate is formed with complementary patterns, the complementary patterns adapted to be engaged with the patterns of the restraining member and to constrain the movement of the restraining member.

6. The chastity device as claimed in claim 4, wherein the pressing unit comprises:
a motor comprising a motor shaft;
a cam mounted to the motor shaft and configured to rotate at a predetermined angle corresponding to a rotational angle of the motor shaft; and
a locking block comprising a first end portion and a second end portion, the first end portion secured to the cam and the second end portion oscillatory coupled to a top side of the locking plate, wherein the second end portion is configured to apply pressing force to constrain a movement of the restraining member.

7. The chastity device as claimed in claim 4, wherein the pressing unit comprises an electromagnetic lock, the electromagnetic lock comprising:
a motor; and
an electromagnetic pressure sheet electromagnetically connected to the motor and configured to apply the pressing force on the locking plate to constrains the movement of the restraining member.

8. The chastity device as claimed in claim 1, wherein the lock structure comprises:
the motor comprising the motor shaft;
a driver shaft comprising a proximal end and a distal end;
a gearbox connecting the motor shaft and the proximal end and configured to control a rotational speed of the driver shaft; and
a limiting member rotatably connected to the distal end of the driver shaft, wherein the limiting member is configured to constrains the movement of the restraining member during a non-use state, and wherein the limiting member pulls the restraining member during a use state.

9. The chastity device as claimed in claim 8, wherein the limiting member is a gear, the gear formed with complementary patterns adapted to be engaged with the patterns of the restraining member.

10. The chastity device as claimed in claim 8, wherein the motor shaft and the driving shaft are orthogonally coupled with each other.

11. The chastity device as claimed in claim 1, wherein the restraining member is formed with at least one notch adapted to tear the restraining member.

12. The chastity device as claimed in claim 1, wherein the restraining member is made up of a flexible material.

13. The chastity device as claimed in claim 1, wherein the main body comprises:
a urination hole configured to allow a urination of the wearer;
a shielding member covering the urination hole at least during non-use state of the urination hole; and
a locking unit coupled to the shielding member and configured to limit an opening and closing of the urination hole.

14. The chastity device as claimed in claim 13, wherein the locking unit is one of a mechanical lock and an electronic lock.

15. The chastity device as claimed in claim 1, further comprising:
a sensing module
an electronic controller; and
a memory unit operably connected to the electronic controller, the memory unit comprising machine-readable instructions, that when executed by the electronic controller, enable the electronic controller to:
generate a report signal indicative of a change in a wearing state of the chastity device, in response to an input signal received from the sensing module installed with the chastity device; and
transmit the report signal to a monitor client device associated with a monitor of the chastity device.

16. The chastity device as claimed in claim 15, wherein the sensing module is associated with at least one of the main body, the lock structure, and the restraining member, and is configured at least to detect the wearing state of the chastity device and measure a tightness of the restraining member.

17. The chastity device as claimed in claim 16, wherein the sensing module comprises a conductive material associated with the restraining member, the conductive material disposed within at least a part of the restraining member at least to build a detection circuit therein, indicative of the wearing state of the chastity device.

18. The chastity device as claimed in claim 1, wherein the restraining member comprises a first end and a second end, one of the first end and the second end configured to pass through the lock structure and coupled to one of the second end and the first end, thereby forming a ring-like structure adapted to be worn by the wearer.

19. The chastity device as claimed in claim 1, wherein the restraining member is an open-close type restraining member.

20. The chastity device as claimed in claim 1, wherein the chastity device is one of a male chastity device or a female chastity device.
